(19) United States
(12) Patent Application Publication
(10) Pub. No.: US 2002/0099400 A1
(43) Pub. Date: Jul. 25, 2002

(54) Cataract removal apparatus

(76) Inventors: John R. Wolf, San Marino, CA (US); Alex Urich, Mission Viejo, CA (US)

Correspondence Address: LAW OFFICES OF WILLIAM L. KLIMA, P.L.C., P.O. Box 2855, Stafford, VA, 22555-2855, US

(21) Appl. No.: 09/765,591

(22) Filed: Jan. 22, 2001

(51) Int. Cl. A61B 17/32

(52) U.S. Cl. 606/169

(57) ABSTRACT

A phacoemulsification apparatus configured for reciprocating a rotary cutting bit, preferably at high frequency. Most preferably, the phacoemulsification apparatus is configured for vibrating and reciprocating the rotary cutting bit in various modes.

### FIELD OF THE INVENTION

An ultrasonic phacoemulsification apparatus for removing the natural crystalline lens of the eye. The ultrasonic phacoemulsification apparatus according to the present invention is configured for rotary and/or reciprocating motion. In a preferred embodiment, a rotary cutting bit is driven by an ultrasonic hand piece.

### BACKGROUND OF THE INVENTION

The conventional phacoemulsification apparatus includes an ultrasonic hand piece and an electrical driver/controller for electronically driving and controlling the ultrasonic hand piece.

The conventional hand piece includes an ultrasonic transducer configured for vibrating an ultrasonic horn. The ultrasonic horn is provided with a mechanical coupler for releasably connecting a phacoemulsification needle thereto. Thus, when assembled the ultrasonic hand piece drives the phacoemulsification needle at a preselected ultrasonic frequency. The ultrasonic needle is rigidly connected to the hand piece, and is configured not to rotate in any manner and vibrate along with the ultrasonic horn of the hand piece. A resilient sleeve (e.g. made of silicone) is removably connected to the hand piece and surrounds the ultrasonic needle except as the tip thereof.

The conventional hand piece is connected to a supply of irrigation fluid with an irrigation line made of tubing, and connected to a pump (e.g. peristaltic, venturi, etc.) by an aspiration line made of tubing. During operation, irrigation fluid flows into the conventional hand piece, and is circulated between a passageway defined between the phacoemulsification needle and sleeve and then out of a tip of the sleeve. The fluid enters the eye and is circulated within the eye, and then aspirated through a fluid passageway provided in the phacoemulsification needle.

### SUMMARY OF THE INVENTION

An object of the present invention is to provide an improved phacoemulsification apparatus.

A second object of the present invention is to provide a phacoemulsification apparatus according to the present invention including a rotary reciprocating hand piece, a rotary cutting bit connected to said hand piece, and a device for driving and controlling the hand piece, the device configured for driving and rotating the hand piece and the rotary cutting bit in a reciprocating motion, preferably in a high frequency reciprocating motion.

A third object of the present invention is to provide a phacoemulsification apparatus according to the present invention wherein the hand piece is an ultrasonic hand piece.

A fourth object of the present invention is to provide a phacoemulsification apparatus according to the present invention wherein the ultrasonic hand piece includes an ultrasonic transducer.

A fifth object of the present invention is to provide a phacoemulsification apparatus according to the present invention wherein said ultrasonic transducer is configured for ultrasonically driving the rotary cutting bit in a high frequency rotary reciprocating motion.

A sixth object of the present invention is to provide a phacoemulsification apparatus according to the present invention wherein the ultrasonic transducer is configured for ultrasonically driving the rotary cutting bit in a high frequency translating motion.

A seventh object of the present invention is to provide a phacoemulsification apparatus according to the present invention wherein the ultrasonic transducer is configured for ultrasonically driving the rotary cutting bit in a high frequency translating motion.

An eighth object of the present invention is to provide a phacoemulsification apparatus according to the present invention including a sleeve surrounding at least a portion of the rotary cutting bit.

A ninth object of the present invention is to provide a phacoemulsification apparatus according to the present invention wherein the sleeve surrounds the rotary cutting bit except for a tip thereof.

A tenth object of the present invention is to provide a phacoemulsification apparatus according to the present invention wherein the rotary cutting bit is provided with a fluid passageway therethrough.

An eleventh object of the present invention is to provide a phacoemulsification apparatus according to the present invention including a sleeve surrounding at least a portion of the rotary cutting bit, a device for providing irrigation fluid into the hand piece, and a device for providing aspiration fluid out of the hand piece.

A twelfth object of the present invention is to provide a phacoemulsification apparatus according to the present invention wherein irrigation fluid is circulated within the hand piece through a first passageway defined by the fluid passageway provided in the rotary cutting bit and out of a tip of the sleeve, and aspiration fluid is then circulated into the tip of the sleeve and through a second passageway defined between the rotary cutting bit and the sleeve.

A thirteenth object of the present invention is to provide a phacoemulsification apparatus according to the present invention wherein irrigation fluid is circulated within the hand piece through a first passageway defined between the rotary cutting bit and the sleeve, and aspiration fluid is then circulated into the tip of the sleeve and through a second passageway defined by the fluid passageway provided in the rotary cutting bit.

A fourteenth object of the present invention is to provide a phacoemulsification apparatus according to the present invention wherein the rotary cutting bit is provided with cutting teeth provided on an end face thereof A fifteenth object of the present invention is to provide a phacoemulsification apparatus according to the present invention wherein the teeth are radially aligned extending from a center portion to an outer portion of the end face of the rotary cutting bit.

The present invention is directed to an improved phacoemulsification apparatus including a rotary cutting bit. The phacoemulsification apparatus according to the present invention is configured to drive the rotary cutting bit in one direction (i.e. clockwise or counter-clockwise) or is two (2) directions (i.e. clockwise and counter-clockwise). Preferably the phacoemulsification apparatus is configured to drive the rotary cutting bit in a reciprocating motion, and more preferably, a high frequency reciprocating motion. Most preferably, the phacoemulsification apparatus is configured to drive the rotary cutting bit with an ultrasonic frequency reciprocating motion.

The reciprocating motion can be translational and/or rotational. Specifically, the rotary cutting bit can be driven to mechanically reciprocate back-and-forth along an axis of its shaft, and/or can be driven to mechanically reciprocate by rotating back-and-forth. The reciprocating motion can be a fixed frequency, randomly changing frequency, and/or non-randomly changing frequency (e.g. cyclic change of frequency). Further, the reciprocating motion can be single frequency or multiple frequency (e.g. 60 Hz with 0.5 mm amplitude vibration provided by mechanical oscillator (i.e. translational) or stepping motor (i.e. rotational) in combination with ultrasonic frequency vibration provided by one or more ultrasonic transducers).

### BRIEF DESCRIPTION OF THE DRAWINGS

FIG. 1 is perspective view of a hand piece for the phacoemulsification apparatus according to the present invention.

FIG. 2 is a side elevational view of the hand piece shown in FIG. 1.

FIG. 3 is a detailed longitudinal cross-sectional view of the rotary cutting bit for the phacoemulsification apparatus according to the present invention.

FIG. 4 is an end elevational view of the rotary cutting bit for the phacoemulsification apparatus according to the present invention.

FIG. 5 is a detailed longitudinal cross-sectional view of an alternative embodiment of a rotary cutting bit for the phacoemulsification apparatus according to the present invention.

FIG. 6 is an end elevational view of the rotary cutting bit shown in FIG. 5.

FIG. 7 is a diagrammatical view showing an electrical stepping motor for driving the rotary cutting bit for the phacoemulsification apparatus according to the present invention.

FIG. 8 is a diagrammatical view of an electrical stepping motor in combination with an ultrasonic transducer configured for both rotating and vibrating in a translational motion the rotary cutting tip for the phacoemulsification apparatus according to the present invention.

FIG. 9 is a diagrammatical view of an electrical stepping motor, first ultrasonic transducer, and a second ultrasonic transducer configured for rotating, vibrating in a translational motion, and vibrating in a rotary motion the rotary cutting bit for the phacoemulsification apparatus according to the present invention.

FIG. 10 is a diagrammatical view showing a possible configuration for the second transducer of FIG. 9, for providing a vibrating rotary motion for the rotary cutting bit for the phacoemulsification apparatus according to the present invention.

FIG. 11 is a diagrammatical view of the phacoemulsification apparatus according to the present invention.

### DETAILED DESCRIPTION OF PREFERRED EMBODIMENTS

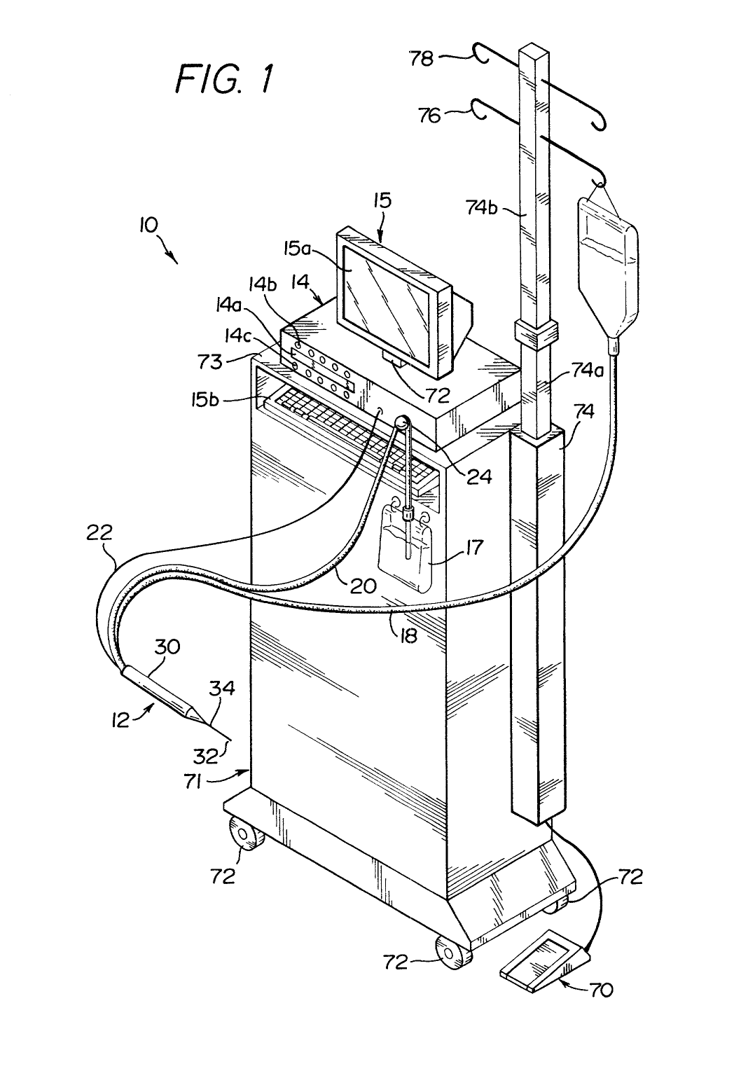

A phacoemulsification apparatus 10 according to the present invention is shown in FIG. 1. The phacoemulsification apparatus 10 is provided with an irrigation supply 16 (e.g. bottle or bag) and a replaceable tubing set including irrigation tubing 18 and aspiration tubing 20. Further, the phacoemulsification apparatus 10 includes the main components of a hand piece 12, control console 14, personal computer 15, foot pedal 70 and movable stand 71.

The control console 14 is provided with a display 14a, and touch control buttons 14b and 14c. The back panel of the control console 14 is provided with a conventional AC power jack. The control console 14 contains a variety of electrical components, including a circuit board and microprocessor controller (not shown).

The personal computer 15 is provided with an integral screen 15a and connected to a remote keyboard 15b. The personal computer 15 is supported above the console 14. The personal computer 15 is programmed to communicate with the control console 14.

A foot pedal 70 is electrically connected to the console 14. The foot pedal 70 is a multi-position type foot pedal configured to control operation of the phacoemulsification apparatus 10.

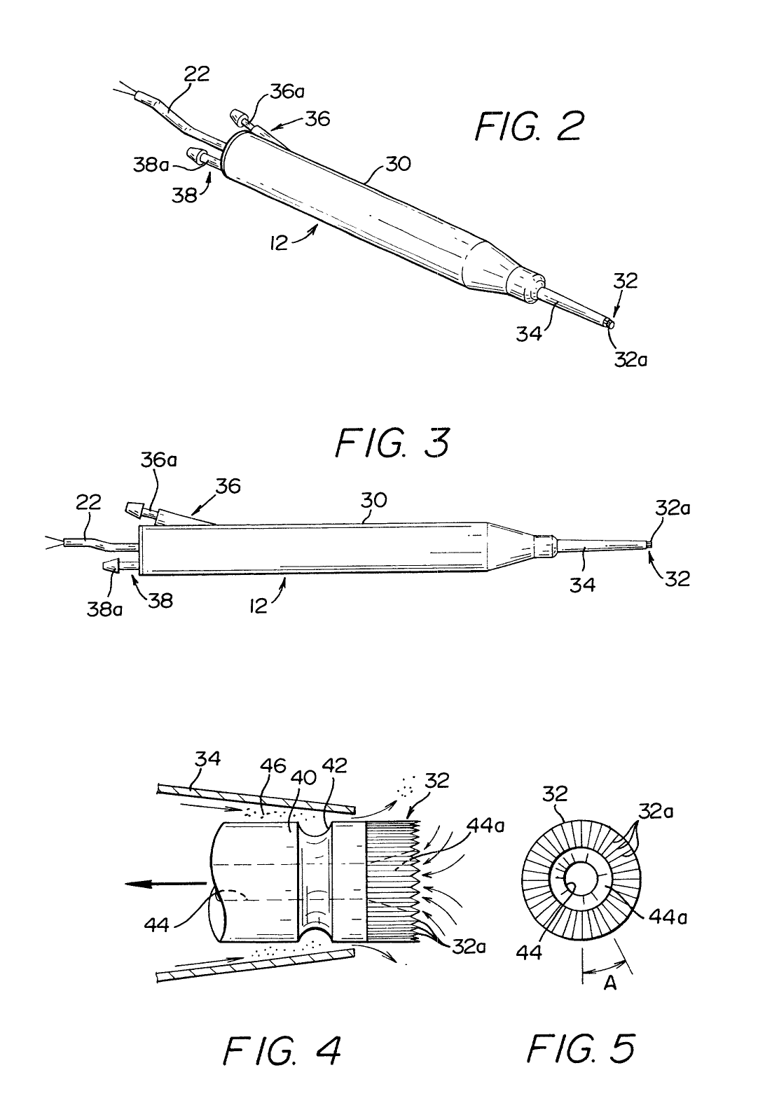

The hand piece 12 for the phacoemulsification apparatus 10 according to the present invention is shown in detail in FIGS. 2 and 3. The hand piece includes a body 30 containing the components for driving a rotary cutting bit 32. A soft resilient sleeve (e.g. made of silicone material) surrounds the rotary cutting bit 32 except for a tip 32a thereof. The sleeve 34 is configured (i.e. sized and shaped) to be inserted and fit within a small incision made in the eye during phacoemulsification surgery.

The hand piece 12 is provided with an irrigation fluid port 36 provided with a tubing connector 36a, and an aspiration fluid port 38 including a tubing connector 38a. An electrical cable 22 connects to hand piece 12 to the console 14 of the phacoemulsification 10, shown in FIG. 1.

The rotary cutting bit 32 is shown in detail in FIGS. 4 and 5. The rotary cutting bit 32 includes a shaft 40 connecting to a drive mechanism enclosed in the body 30 of the hand piece 12. The shaft 40 is provided with an annular recess 42 to provide a fluid bearing between the inner surface of the sleeve 34 and the outer surface of the rotary cutting bit 32 along shaft 36 to prevent wear or damage to the inner surface of the sleeve 34 by the rotating movement of the rotary cutting bit 32. The rotating cutting bit 32 is provided with a fluid passageway 44. In operation, irrigation fluid is provided along irrigation tubing 18 to irrigation fluid port 36 of the hand piece 12, and flows through the hand piece 12 and then through an annular fluid passageway 46 defined between the shaft 40 of the rotary cutting bit 32 and the sleeve 34. The irrigation fluid then flows into the eye and is circulated, and then drawn in by suction into the fluid passageway 44 of the rotating cutting bit 32.

The rotary cutting bit 32 is provided with a plurality of radially extending teeth 32a, shown in FIG. 5. The individual teeth 32a are radially oriented and extend from a center of the rotary cutting bit 32 to an outer peripheral edge of the rotary cutting bit 32. The fluid passageway 44 includes a wider inwardly tapering fluid passageway 44a. The tapering fluid passageway 44a provides somewhat of a venturi effect to help facilitate the circulation and aspiration of fluid and lens particles resulting from the phacoemulsification of the natural crystalline lens.

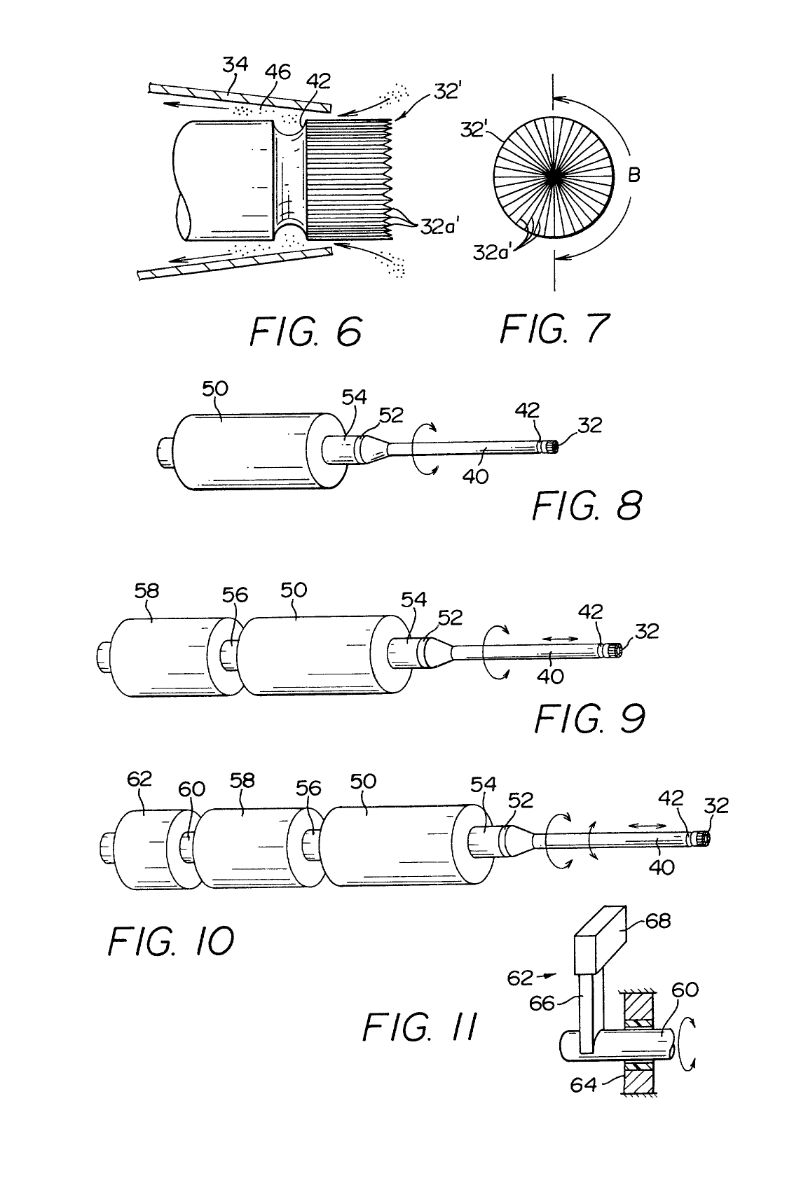

In an alternative embodiment shown in FIGS. 6 and 7, a rotary cutting bit 32′ not provided with a fluid passageway, is used with the phacoemulsification apparatus 10 according to the present invention. In this embodiment, a side port incision is provided in the eye and irrigation fluid is provided through a separate needle or cannula into the side port in the eye. The aspiration of fluid and particles from the natural crystalline lens are aspirated through the annular fluid passageway 46. Thus, in this embodiment, a fluid passageway 44 of the embodiment shown in FIGS. 4 and 5 is not required.

The hand piece 12 of the phacoemulsification apparatus 10 according to the present invention can be configured in various manners. Specifically, the hand piece 12 can be configured to provide the following modes of operation:

1) one direction (e.g. clockwise or counterclockwise) rotary motion of the rotary cutting bit 32;

2) two (2) direction (i.e. clockwise and counterclockwise) rotary motion of the rotary cutting bit 32;

3) ultrasonic translational vibration (i.e. ultrasonic vibration along the longitudinal axis) of the rotary cutting bit 32;

4) ultrasonic rotary vibration (i.e. rotational movement) of the rotary cutting bit 32; and

5) any combination of the above motions.

These various modes of operation of the rotary cutting bit 32 can be provided by various electrical components or assemblies within the body 30 of the hand piece 12.

In the embodiments shown in FIG. 8, an electrical rotary stepping motor 50 is connected to the shaft 40 of the rotary cutting bit 32 by mechanical coupling 52 (e.g. internal threaded shaft 54 of rotary stepping motor 50 and external threaded shaft of the shaft 40). The rotary stepping motor 50 is configured to incrementally rotate the rotary cutting bit in one (1) direction (i.e. clockwise or counterclockwise) or two (2) directions (clockwise and counterclockwise). The electronic controller in the console 14 can be programmed so that the magnitude of rotation (i.e. number of degrees and minutes of rotation) can be fixed or varied. The programming can be such that the magnitude can be changed randomly or non-randomly (e.g. cyclically). For example, the magnitude can increase a fixed amount or variable amount each cycle until a preset limit and then return to its initially set lower limit. As shown in FIG. 5, the angle A has a small magnitude (i.e. approximately 10 degrees) versus the larger magnitude shown in FIG. 7 (i.e. approximately 180 degrees). In these embodiments, the rotary cutting bit 32 is rotated back and forth through the angles shown.

In the embodiment shown in FIG. 9, the rotary stepping motor 50 is mechanically coupled through shaft 56 to ultrasonic transducer 58 configured to ultrasonically vibrate the rotary cutting bit 32 in a translational motion (i.e. vibrating back and forth along the longitudinal axis of the shaft 40 of the rotary cutting bit 32).

In the embodiment shown in FIG. 10, the rotary stepping motor 50 is mechanically coupled via shaft 56 to ultrasonic transducer 58, which in turn is mechanically coupled via shaft 60 to ultrasonic transducer 62. The ultrasonic transducer 62 is configured to provide ultrasonic vibrating rotary movement to the shaft 40 of the rotary cutting tip 32. For example, the ultrasonic transducer 62 can be configured as shown in FIG. 11. Specifically, in this embodiment the shaft 60 is supported by a bearing 64 with an arm 66 connecting the shaft 62 and ultrasonic vibrator 68. When the ultrasonic vibrator 64 is activated, an ultrasonic vibrating rotary moment is provided along arm 66 to shaft 60. Thus, an ultrasonic vibrating torsional force is applied along the shaft 60 in a back and forth motion. The frequency and magnitude of the ultrasonic vibrator 68 can be controlled via a program to adjust and change the magnitude and direction of the ultrasonic vibration motion of the shaft 60 mechanically coupled to shaft 40 of the rotating cutting bit 32.

What is claimed is:

1. A phacoemulsification apparatus, comprising:
a rotary reciprocating hand piece;
a rotary cutting bit connected to said hand piece; and
a device for driving and controlling said hand piece, said device configured for driving and rotating said hand piece and said rotary cutting bit in a high frequency reciprocating motion.

2. An apparatus according to claim 1, wherein said hand piece is an ultrasonic hand piece.

3. An apparatus according to claim 2, wherein said ultrasonic hand piece includes an ultrasonic transducer.

4. An apparatus according to claim 3, wherein said ultrasonic transducer is configured for ultrasonically driving said rotary cutting bit in a high frequency rotary reciprocating motion.

5. An apparatus according to claim 3, wherein said ultrasonic transducer is configured for ultrasonically driving said rotary cutting bit in a high frequency translating motion.

6. An apparatus according to claim 4, wherein said ultrasonic transducer is configured for ultrasonically driving said rotary cutting bit in a high frequency translating motion.

7. An apparatus according to claim 1, including a sleeve surrounding at least a portion of said rotary cutting bit.

8. An apparatus according to claim 7, wherein said sleeve surrounds said rotary cutting bit except for a tip thereof.

9. An apparatus according to claim 1, wherein said rotary cutting bit is provided with a fluid passageway therethrough.

10. An apparatus according to claim 9, including:
a sleeve surrounding at least a portion of said rotary cutting bit;
a device for providing irrigation fluid into said hand piece; and
a device for providing aspiration fluid out of said hand piece.

11. An apparatus according to claim 10, wherein irrigation fluid is circulated within said hand piece through a first passageway defined by said fluid passageway provided in said rotary cutting bit and out of a tip of said sleeve, and aspiration fluid is then circulated into said tip of said sleeve and through a second passageway defined between said rotary cutting bit and said sleeve.

12. An apparatus according to claim 10, wherein irrigation fluid is circulated within said hand piece through a first passageway defined between said rotary cutting bit and said sleeve, and aspiration fluid is then circulated into said tip of said sleeve and through a second passageway defined by said fluid passageway provided in said rotary cutting bit.

13. An apparatus according to claim 1, wherein said rotary cutting bit is provided with cutting teeth provided on an end face thereof.

14. An apparatus according to claim 13, wherein said teeth are radially aligned extending from a center portion to an outer portion of said end face of said rotary cutting bit.
